(19) United States
(12) Patent Application Publication
(10) Pub. No.: US 2007/0161903 A1
(43) Pub. Date: Jul. 12, 2007

(54) ARRAY-TYPE ULTRASONIC PROBE AND ULTRASONIC DIAGNOSTIC APPARATUS

(76) Inventors: Yohachi Yamashita, Yokohama-shi (JP); Yasuharu Hosono, Yokohama-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/533,150

(22) Filed: Sep. 19, 2006

(30) Foreign Application Priority Data
Jan. 11, 2006 (JP) 2006-003966

(51) Int. Cl. A61B 8/14

(52) U.S. Cl. 600/459

(57) ABSTRACT

An array-type ultrasonic probe includes a member, a plurality of channels arranged on the member with spaces and each having a piezoelectric element and at least three acoustic matching layers formed on the piezoelectric element, and an acoustic lens formed in a manner to cover at least the surface of the uppermost acoustic matching layer of each channel. The uppermost acoustic matching layer contains a denatured polyether resin and an another resin having an acoustic impedance higher than that of the denatured polyether resin, the uppermost acoustic matching layer having an acoustic impedance of 1.6 to 2.5 MRayls at 25° C.

### CROSS-REFERENCE TO RELATED APPLICATIONS

This application is based upon and claims the benefit of priority from prior Japanese Patent Application No. 2006-003966, filed Jan. 11, 2006, the entire contents of which are incorporated herein by reference.

### BACKGROUND OF THE INVENTION

1. Field of the Invention

The present invention relates to an array-type ultrasonic probe for transmitting an ultrasonic signal to an object and for receiving an ultrasonic signal reflected from the object, and an ultrasonic diagnostic apparatus comprising the array-type ultrasonic probe.

2. Description of the Related Art

A medical ultrasonic diagnostic apparatus or a medical ultrasonic image inspecting apparatus transmits an ultrasonic signal to an object and, by receiving a reflection signal (echo signal) reflected from the object, forms an image showing the intend state of the object. In the medical ultrasonic diagnostic apparatus or the medical ultrasonic image inspecting apparatus, an electronic array-type ultrasonic probe, which performs the function of transmitting and receiving the ultrasonic signal, is mainly used.

The array-type ultrasonic probe comprises a backing member, a plurality of channels arranged on the backing member with spaces in a manner to form an array, and an acoustic lens bonded to the channels. Each of the plural channels comprises a piezoelectric element formed on the backing member and an acoustic matching layer formed on the piezoelectric element. The piezoelectric element noted above includes a piezoelectric body consisting of, for example, a zirconium lead titanate (PZT) series piezoelectric ceramic material or a relaxer series single crystalline material and electrodes formed on both surfaces of the piezoelectric body. Incidentally, trenches are formed in some cases in a surface region of the backing member in a manner to correspond to the spaces between the adjacent channels.

In performing the diagnosis, the piezoelectric element of each channel is driven under the state that the array-type ultrasonic probe is placed such that the acoustic lens included in the ultrasonic probe is allowed to abut against an object so as to permit an ultrasonic signal to be transmitted from the front surface of the piezoelectric element to the object. The ultrasonic signals are converged on a prescribed position within the object by the electronic focus and the focus of the acoustic lens in accordance with the driving timing of the piezoelectric element. In this stage, it is possible to transmit the ultrasonic signals into a prescribed range within the object by controlling the driving timing of the piezoelectric element, and an ultrasonic image (tomographic image) within the prescribed range can be obtained by receiving and processing the echo signal given from the object. When the piezoelectric element of the ultrasonic probe is driven, an ultrasonic signal is also released to the rear of the piezoelectric element. Therefore, it is advisable to arrange the backing member on the back surface of the piezoelectric element of each channel so as to permit the ultrasonic signal released to the rear to be absorbed (attenuated) by the backing member. As a result, it is possible to avoid the adverse effect that the normal ultrasonic signal is transmitted into the object together with the ultrasonic signal (reflection signal) reflected from the rear of the piezoelectric element.

The acoustic matching layer of a single layer structure, a double layer structure or a multi-layered structure including three or more layers is known in the art. JP-A 2005-198261 (KOKAI) discloses an array-type ultrasonic probe which comprises an acoustic matching layer having three or more layers for broadening the bandwidth.

In the driving stage of the array-type ultrasonic probe, the energy of the ultrasonic wave radiated from the piezoelectric elements of the plural channels is partly absorbed and attenuated by the acoustic matching layer and the acoustic lens. In this stage, the energy of the ultrasonic wave is partly converted into heat, with the result that, in the ultrasonic probe for, for example, a cardiac probe, it is possible for the temperature of the acoustic matching layer to be elevated to 60° C. or more. Further, during use of the ultrasonic probe, a considerably high pressure is applied through the acoustic lens to the acoustic matching layer. Because of the thermal effect given above, the acoustic lens and the acoustic matching layer are made different from each other in the degree of the thermal expansion. In addition, the mechanical pressure is applied to the acoustic matching layer through the acoustic lens. It follows that peeling tends to occur between the acoustic lens and the uppermost acoustic matching layer and between the uppermost acoustic matching layer and the acoustic matching layer immediately below the uppermost acoustic matching layer. As a result, a nonuniformity in the sensitivity is generated within the ultrasonic probe so as to lower the reliability of the ultrasonic probe. In the extreme case, the function of the ultrasonic probe is caused to be stopped.

The characteristics etc. of the acoustic matching layer used in the ultrasonic probe are exemplified in detail in JP-A 2005-198261 referred to above. For example, it is described that a material having an acoustic impedance of 1.5 to 3.5 MRayls closer to the acoustic impedance (1.4 to 1.6 MRayls) of an object, e.g., a living body, is used for forming the uppermost acoustic matching layer in contact with the acoustic lens among the plural acoustic matching layers.

It was customary in the past to use a material based on polyurethane, polyethylene, a silicone rubber or an epoxy resin for forming the acoustic matching layer. To be more specific, it is disclosed in JP-A 10-75953 (KOKAI) that an acoustic matching layer having a low heat conductivity is used as one of the acoustic matching layers arranged between the piezoelectric element and the surface of an object, e.g., the surface of a living body, so as to decrease the amount of heat transmitted from the heat generating body arranged within the ultrasonic probe to the surface of the object, i.e., the surface of a living body. It is disclosed that the acoustic matching layer having a low heat conductivity is formed of a material prepared by dispersing fine particles of a material having a low heat conductivity, such as a silicone resin, in a base material, such as an epoxy resin. Also, an acoustic matching layer in which a polyethylene fiber or a carbon fiber is loaded in polyurethane is disclosed in “Toshio Kondo and Hiroyuki Fujimoto, Proceedings 2003 IEEE Ultrasonic Symposium p. 1318-1321” and “Toshio Kondo, Hiroyuki Mitsuyoshi Kitatuji and Mikio Izumi, Proceedings 2004 IEEE Ultrasonic Symposium p. 1659-1662”.

However, none of the materials quoted above satisfies all the requirements of the acoustic matching layer including a low attenuation, excellent dicing machinability excellent heat resistance, excellent bonding properties between the upper and lower layers and appropriate acoustic impedance.

### BRIEF SUMMARY OF THE INVENTION

According to a first aspect of the present invention, there is provided an array-type ultrasonic probe, comprising:

a member;

a plurality of channels arranged on the member with spaces and each having a piezoelectric element and at least three acoustic matching layers formed on the piezoelectric element; and

an acoustic lens formed in a manner to cover at least the surface of the uppermost acoustic matching layer of each channel,

wherein the uppermost acoustic matching layer contains a denatured polyether resin and an another resin having an acoustic impedance higher than that of the denatured polyether resin, the uppermost acoustic matching layer having an acoustic impedance of 1.6 to 2.5 MRayls at 25° C.

According to a second aspect of the present invention, there is provided an array-type ultrasonic probe, comprising:

a member;

a plurality of channels arranged on the member with spaces and each having a piezoelectric element and at least three acoustic matching layers formed on the piezoelectric element; and

an acoustic lens formed in a manner to cover at least the surface of the uppermost acoustic matching layer of each channel,

wherein the lowermost acoustic matching layer in contact with the piezoelectric element has an acoustic impedance of 10 to 15 MRayls at 25° C., the intermediate acoustic matching layer has an acoustic impedance of 2.7 to 8 MRayls at 25° C., and the uppermost acoustic matching layer in contact with the acoustic lens contains a denatured polyether resin and an another resin having an acoustic impedance higher than that of the denatured polyether resin, the uppermost acoustic matching layer having an acoustic impedance of 1.6 to 2.5 MRayls at 25° C.

According to a third aspect of the present invention, there is provided an array-type ultrasonic probe, comprising:

a member;

a plurality of channels arranged on the member with spaces and each having a piezoelectric element and at least four acoustic matching layers formed on the piezoelectric element; and

an acoustic lens formed in a manner to cover at least the surface of the uppermost acoustic matching layer of each channel,

wherein the lowermost acoustic matching layer in contact with the piezoelectric element has an acoustic impedance of 14 to 20 MRayls at 25° C., the second acoustic matching layer in contact with the lowermost acoustic matching layer has an acoustic impedance of 7 to 12 MRayls at 25° C., the third acoustic matching layer in contact with the second acoustic matching layer has an acoustic impedance of 3 to 5 MRayls at 25° C., and the uppermost acoustic matching layer in contact with the acoustic lens contains a denatured polyether resin and an another resin having an acoustic impedance higher than that of the denatured polyether resin, the uppermost acoustic matching layer having an acoustic impedance of 1.6 to 2.5 MRayls at 25° C.

According to a fourth aspect of the present invention, there is provided an ultrasonic diagnostic apparatus, comprising:

an array-type ultrasonic probe according to any of the first to third aspects; and

an ultrasonic probe controller connected to the ultrasonic probe through a cable.

### BRIEF DESCRIPTION OF THE SEVERAL VIEWS OF THE DRAWING

FIG. 1 is an oblique view showing the construction of a gist portion of an array-type ultrasonic probe according to an embodiment;

FIG. 2 is a partial cross-sectional view showing the construction of the gist portion of the ultrasonic probe shown in FIG. 1;

FIG. 3 is a cross-sectional view schematically showing the construction of a third acoustic matching layer incorporated in the array-type ultrasonic probe according to an embodiment;

FIG. 4 is a cross-sectional view schematically showing the construction of another third acoustic matching layer incorporated in the array-type ultrasonic probe according to an embodiment; and

FIG. 5 schematically shows the construction of an ultrasonic diagnostic apparatus according to an embodiment.

### DETAILED DESCRIPTION OF THE INVENTION

An array-type ultrasonic probe and an ultrasonic diagnostic apparatus according to an embodiment of the present invention will now be described in detail with reference to the accompanying drawings.

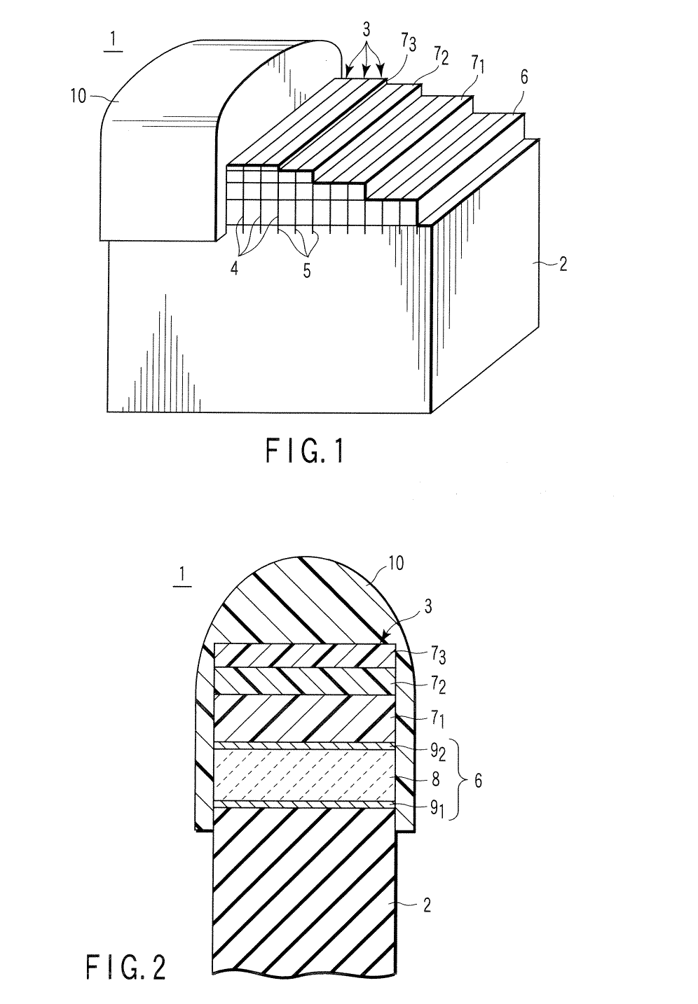

FIG. 1 is an oblique view showing the construction of a gist portion of an array-type ultrasonic probe 1 according to an embodiment of the present invention, and FIG. 2 is a partial cross-sectional view showing the construction of the array-type ultrasonic probe 1 shown in FIG. 1.

As shown in the drawings, the array-type ultrasonic probe 1 comprises a member 2 (e.g., a backing member). The member 2 is not limited to a backing member. It is possible for the member 2 to represent a backing member including a flexible printed circuit (FPC). A plurality of channels 3 are arranged on the backing member 2. These channels 3 are arranged with a prescribed space 4 given therebetween. A plurality of trenches 5 are formed in the backing member 2 in a manner to correspond to the spaces 4 between the adjacent channels 3. Incidentally, it is possible to load a relatively soft resin having, for example, a low acoustic impedance and a high attenuation capability, such as a silicone rubber, in the space 4 between the adjacent channels 3 so as to maintain a high mechanical strength of the channel 3. Also, it is possible for the particular resin to be loaded not only in the space 4 but also in the trench 5 formed in a surface region of the backing member 2 so as to be positioned below the space 4.

Each of the channels 3 comprises a piezoelectric element 6 and at least three acoustic matching layers, e.g., first to third acoustic matching layers 7₁ to 7₃, which are formed on the piezoelectric element 6. As shown in FIG. 2, the piezoelectric element 6 comprises a piezoelectric body 8 and first and second electrodes 9₁, 9₂ formed on both surfaces of the piezoelectric body 8. The piezoelectric body 8 is made of, for example, a lead zirconium titanate (PZT) series piezoelectric ceramic material or a relaxor series single crystal material. The first electrode 9₁ of the piezoelectric element 6 is bonded and fixed to the backing member 2 by using, for example, an epoxy-based resin adhesive layer (not shown). The first acoustic matching layer 7₁ is bonded and fixed to the second electrode 9₂ of the piezoelectric element 6 by using, for example, an epoxy-based resin adhesive layer (not shown). The second acoustic matching layer 7₂ is bonded and fixed to the first acoustic matching layer 7₁ by using, for example, an epoxy-based resin adhesive layer (not shown). Further, the third acoustic matching layer 7₃ (the uppermost acoustic matching layer) is bonded and fixed to the second acoustic matching layer 7₂ by using an epoxy-based resin adhesive layer (not shown).

An acoustic lens 10 is formed to cover the upper surface of the uppermost acoustic matching layer 7₃ of the channel 3, the side surfaces of the third, second and first acoustic matching layers 7₃, 7₂, 7₁, and the side surface of that portion of the backing member 2 which is positioned in the vicinity of the piezoelectric element 6. It should be noted that the acoustic lens 10 is bonded and fixed to the upper surface of the third acoustic matching layer 7₃ by using a rubber-based adhesive layer (not shown). It is desirable for the rubber-based adhesive to be formed of a silicone-based adhesive having an acoustic impedance of 1.3 to 1.8 MRayls at 25° C.

The backing member 2, the plural channels 3 and the acoustic lens 10 are housed in a case (not shown). Also housed in the case is a signal processing circuit (not shown) including a control circuit for controlling the driving timing of the piezoelectric element 6 of each channel 3 and an amplifier circuit for amplifying the signal received by the piezoelectric element 6. A signal line and a ground lead (not shown) are connected to the first and second electrodes 9₁ and 9₂ of the piezoelectric element 6, respectively, and are drawn to the outside from the case on the opposite side of the acoustic lens 10 so as to be connected to a control circuit (not shown). Incidentally, it is possible for the signal line to be connected to the second electrode 9₂ of the piezoelectric element 6 and for the ground lead to be connected to the junction between the second acoustic matching layer 7₂ and the third acoustic matching layer 7₃.

In the array-type ultrasonic probe of the construction described above, a voltage is applied between the first and second electrodes 9₁ and 9₂ of the piezoelectric element 6 included in each of the channels 3 so as to cause the piezoelectric body 8 to resonate. As a result, an ultrasonic wave is radiated (transmitted) through the first to third acoustic matching layers 7₁, 7₂, 7₃ included in each channel 3 and the acoustic lens 10. When the echo signal is received, the piezoelectric body 8 of the piezoelectric element 6 in each channel 3 is vibrated by the ultrasonic wave received through the acoustic lens 10 and the first to third acoustic matching layers 7₁, 7₂, 7₃ included in each channel 3, and the vibration of the piezoelectric body 8 is electrically converted into a signal so as to obtain an image.

The third acoustic matching layer (the uppermost acoustic matching layer) 7₃ contains a denatured polyether resin and an another resin having an acoustic impedance higher than that of the denatured polyether resin and exhibits an acoustic impedance of 1.6 to 2.5 MRayls at 25° C.

The denatured polyether resin noted above includes, for example, an allyl denatured polyether, a silyl denatured polyether and a polyether polyol. The term “denatured” denotes, for example, the sililation or the allylation.

The another resin having an acoustic impedance higher than that of the denatured polyether resin includes, for example, an epoxy-based resin, an acrylic resin, a polystyrene-based resin, a polymethyl methacrylate (PMMA)-based resin, and a fluorine-based resin. Among these resins, it is particularly desirable to use as the another resin the epoxy-based resin because a liquid resin can be obtained easily and the epoxy-based resin exhibits a high compatibility with the denatured polyether.

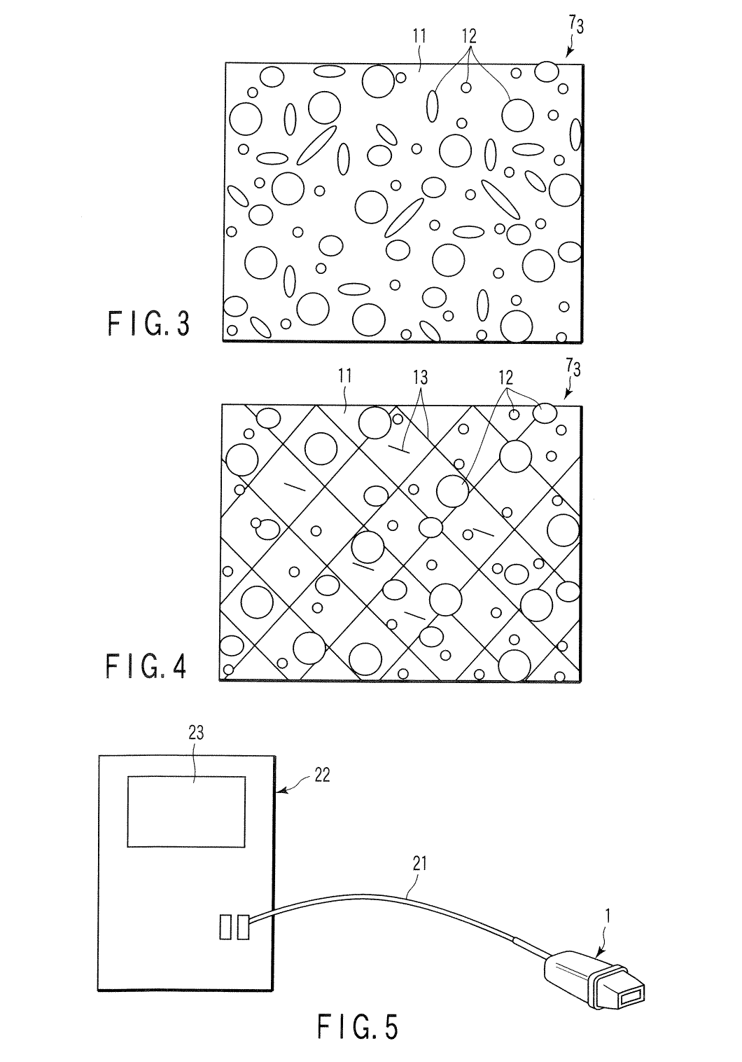

It is desirable that resin particles (e.g., epoxy resin particles) as an another resin having an acoustic impedance higher than that of the denatured polyether resin are dispersed in the denatured polyether resin matrix in an amount of 10 to 60% by volume based on the sum of the matrix and the epoxy resin particles. It is desirable for the epoxy resin particles to have an average particle diameter of 1 to 5 μm. The uppermost acoustic matching layer 7₃ formed of the polymer alloy noted above is constructed as shown in, for example, FIG. 3. As shown in the drawing, epoxy resin particles 12 that are shaped to be, for example, spherical or elliptical are dispersed in a denatured polyether resin matrix 11. The uppermost acoustic matching layer noted above exhibits a longitudinal wave acoustic velocity of 1,300 to 1,800 m/s at 25° C. If the dispersed amount of the epoxy resin particles is smaller than 10% by volume, it is difficult to improve effectively the adhesivity of the epoxy-based adhesive to the acoustic matching layer and the machinability of the acoustic matching layer. On the other hand, if the dispersed amount of the epoxy resin particles exceeds 60% by volume, the attenuation of the ultrasonic wave is rapidly increased, and the acoustic velocity is increased to exceed 1,800 m/s. As a result, it is possible for the value of the acoustic impedance to be increased to exceed 2.5 MRayls. It follows that it is difficult to obtain an acoustic matching layer having a low acoustic impedance. It is more desirable for the dispersed amount of the epoxy resin particles to be 20 to 50% by volume based on the sum of the matrix and the epoxy resin particles.

It is desirable for the third acoustic matching layer (the uppermost acoustic matching layer) 7₃ to further contain an inorganic filler having a density not higher than 6 g/cm³ in an amount not larger than 30% by volume based on the sum of the polymer alloy and the inorganic filler. The acoustic matching layer satisfying the particular requirement exhibits a attenuation not higher than 8 dB/cm MHz, which is measured at 25° C. and at 5 MHz, and a damping capacity index, i.e., the product between the attenuation and the acoustic velocity, which is not larger than 1,500 m/s·dB/mm MHz. Also, the acoustic matching layer having the inorganic filler added thereto permits effectively improving the working strength and heat resistance, compared with the acoustic matching layer not having the inorganic filler added thereto. If the content of the inorganic filler exceeds 30% by volume, it is difficult to load the inorganic filler to the polymer alloy so as to make it difficult to obtain an acoustic matching layer having a uniform composition. In addition, it is possible for the attenuation of the acoustic matching layer to be increased. It is more desirable for the addition amount of the inorganic filler to be 5 to 15% by volume based on the sum of the polymer alloy and the inorganic filler.

The inorganic filler is, for example, powdery or fibrous. It is possible to permit the powdery or fibrous inorganic filler to be contained in the polymer alloy singly or in the form of a mixture.

The powdery inorganic filler includes, for example, zinc oxide powder, zirconium oxide powder, alumina powder, silica powder such as aerosil silica, titanium oxide powder, silicon carbide powder, aluminum nitride power, carbon powder and boron nitride powder. The powdery inorganic filler can be used singly or in the form of a mixture. It is desirable for the powdery inorganic filler to have an average particle diameter not larger than 0.5 μm, more preferably, not larger than 0.1 μm. Where the uppermost acoustic matching layer 7₃ is formed of a polymer alloy having such a fine powdery inorganic filler dispersed therein, it is possible to decrease further the attenuation of the acoustic matching layer.

The fibrous inorganic filler includes, for example, a carbon fiber, a silicon carbide fiber, a zinc oxide fiber, an alumina fiber and a glass fiber. The fibrous inorganic filler can be used singly or in the form of a mixture. The uppermost acoustic matching layer 7₃ formed of the particular polymer alloy is constructed as shown in, for example, FIG. 4. As shown in the drawing, the epoxy resin particles 12, which are shaped to be, for example, spherical, and an inorganic fiber 13 are dispersed in the denatured polyether resin matrix 11.

It is particularly desirable to use a glass fiber as the fibrous inorganic filler. The glass fiber includes, for example, a quartz glass fiber and a soda-lime glass fiber. Also, it is possible to impart electrical conductivity to the acoustic matching layer by using a conductive carbon fiber as the fibrous inorganic filler. The carbon fibers of various grades can be used in the embodiment including, for example, a pitch-based carbon fiber and a PAN-based carbon fiber. It is also possible to use carbon nano tubes as the carbon fiber. Incidentally, the fibrous inorganic filler is not limited to that formed of a single kind of the material. For example, it is possible for the surface of the carbon fiber to be covered with a diamond film or a metal film by the CVD method or to be covered with resin.

It is desirable for the fibrous inorganic filler to have a diameter not larger than 10 μm and a length at least 5 times as much as the diameter. Where a fibrous inorganic filler of the size given above is used for preparing an acoustic matching layer, it is possible to decrease easily the attenuation of the acoustic matching layer to 6 dB/cm MHz or less, which is measured at 5 MHz, while decreasing the mixing amount of the fibrous inorganic filler. It follows that it is possible for the ultrasonic probe to transmit and receive ultrasonic signals without causing the ultrasonic signals to be deteriorated by the acoustic matching layer. Also, sufficient strength required in the stage of the dicing treatment can be imparted to the acoustic matching layer. Further, it is possible to further improve the heat resistance and the machinability in the stage of the dicing treatment of the acoustic matching layer. Particularly, it is possible to provide an acoustic matching layer exhibiting a further decreased attenuation by using a fibrous inorganic filler having a diameter no grater than 5 μm. Also, it is possible to obtain an acoustic matching layer that exhibits a further improved heat resistance and machinability by using a fibrous inorganic filler having a length at least 20 times the diameter.

In the acoustic matching layer of the three-layer structure, it is desirable for the acoustic matching layers other than the uppermost acoustic matching layer 7₃, i.e., for the lowermost acoustic matching layer (i.e., the first acoustic matching layer 7₁ that is in contact with the piezoelectric element 6) to have an acoustic impedance of 10 to 15 MRayls at 25° C. and for the intermediate acoustic matching layer (i.e., the second acoustic matching layer 7₂) to have an acoustic impedance of 2.7 to 8 MRayls at 25° C. In the acoustic matching layer having the particular three layers structure, the thickness of each of these acoustic matching layers is changed depending on the acoustic velocity. The uppermost acoustic matching layer 7₃ has a standard thickness of λ/4, where λ denotes the wavelength of the ultrasonic wave. To be more specific, it is desirable for the uppermost acoustic matching layer 7₃ to have a thickness falling within a range of 30 to 200 μm.

How to manufacture the particular acoustic matching layer will now be described in the following.

In the first step, the raw materials consisting of, for example, 100 parts by weight of a liquid denatured polyether resin, 10 to 200 parts by weight of a liquid bisphenol A type epoxy resin (an another resin), 1 to 5 parts by weight of a bisphenol type antioxidant, 0.5 to 2 parts by weight of diphenyl silane diol, 1 to 3 parts by weight of an organic tin compound, 1 to 10 parts by weight of 2,4,6-tris (dimethylamino methyl) phenol epoxy resin, which is used as a curing agent, and 0.2 to 1 part by weight of distilled water are sufficiently mixed. Then, the mixture is put in a container made of polyethylene and de-gassed, followed by curing the de-gassed mixture at room temperature to 50° C. for 72 hours so as to manufacture a desired acoustic matching layer.

Incidentally, in manufacturing the acoustic matching layer, it is possible to mix the powdery inorganic filler referred to above in the raw materials. Where the viscosity of the composition obtained by the mixing is increased, it is possible to lower the viscosity by using an organic solvent such as normal hexane or toluene. Further, it is possible to permit the mixed composition to be impregnated with the fibrous inorganic filler referred to above by means of, for example, the vacuum impregnation before the curing stage of the composition.

Incidentally, in the embodiment described above, the acoustic matching layer was of a three-layers structure including the first to third acoustic matching layers 7₁, 7₂ and 7₃ as shown in FIG. 1. However, it is also possible for the acoustic matching layer to have a four-layers structure.

Where the acoustic matching layer comprises four layers, it suffices for the uppermost acoustic matching layer to be constructed like the third acoustic matching layer (i.e., the uppermost acoustic matching layer) 7₃ referred to above. In this case, the three intermediate acoustic matching layers are formed appropriately by using appropriate materials such that the acoustic impedance of each of these intermediate acoustic matching layers approaches to the acoustic impedance of the uppermost acoustic matching layer.

In an array-type ultrasonic probe including the acoustic matching layer of a four-layers structure, it is desirable for the lowermost acoustic matching layer in contact with the piezoelectric element to have an acoustic impedance of 14 to 20 MRayls at 25° C., for the second acoustic matching layer to have an acoustic impedance of 7 to 12 MRayls at 25° C., for the third acoustic matching layer to have an acoustic impedance of 3 to 5 MRayls at 25° C., and for the uppermost acoustic matching layer in contact with the acoustic lens to contain a denatured polyether resin and an another resin having an acoustic impedance higher than that of the denatured polyether resin. It is also desirable for the uppermost acoustic matching layer to have an acoustic impedance of 1.6 to 2.5 MRayls at 25° C.

How to manufacture an ultrasonic probe according to an embodiment will now be described in the following.

In the first step, the piezoelectric element described above and the first to third acoustic matching layers are laminated on a backing member one upon the other in the order mentioned, with an epoxy resin-based adhesive having a low viscosity interposed between the adjacent laminated layers. Then, the laminate structure is heated for about one hour at, for example, 120° C. so as to cure the epoxy resin-based adhesive, thereby permitting the piezoelectric element, the first acoustic matching layer, the second acoustic matching layer, and the third acoustic matching layer to be bonded and fixed to each other.

In the next step, the laminate structure is cut from the third acoustic matching layer toward the backing member in a width of, for example, 50 to 200 μm by using, for example, a dicing blade, so as to divide the laminate structure into a plurality of arrayed sections, thereby forming a plurality of channels each having the first to third acoustic matching layers. In this stage, trenches corresponding to the spaces between the adjacent channels are formed in the surface region of the backing member. Then, a relatively soft resin having, for example, a low acoustic impedance and a high attenuation capability, such as a silicon rubber, is loaded in the space between the adjacent channels, as required, so as to maintain a sufficient mechanical strength of each channel. Further, an acoustic lens is bonded and fixed to the third acoustic matching layer in each channel by using a silicone rubber-based adhesive layer. Still further, the backing member, the plural channels and the acoustic lens are housed in a case so as to manufacture an ultrasonic probe.

Incidentally, in manufacturing the third acoustic matching layer, a liquid epoxy polyether resin is prepared by mixing, for example, a denatured polyether resin and an epoxy resin used as an another resin. Then, a glass fiber or a carbon fiber is impregnated with the liquid epoxy polyether resin so as to manufacture the third acoustic matching layer.

The description given above is directed to the manufacturing method of an ultrasonic probe in which the acoustic matching layer has a three-layers structure. However, an ultrasonic probe in which the acoustic matching layer has a four-layers structure can also be manufactured similarly. To be more specific, the ultrasonic probe in which the acoustic matching layer has a four-layers structure can be manufactured as above, except that the piezoelectric element described above and the acoustic matching layer of the four-layers structure are laminated on the backing member.

An ultrasonic diagnostic apparatus comprising the ultrasonic probe according to the embodiment will now be described with reference to FIG. 5.

A medical ultrasonic diagnostic apparatus (or a medical ultrasonic wave inspecting apparatus), which transmits an ultrasonic signal to an object and receives a reflection signal (echo signal) given from the object so as to form an image of the object comprises an array-type ultrasonic probe 1 capable of transmitting-receiving ultrasonic signals. The ultrasonic probe 1 is constructed as shown in FIGS. 1 and 2. As shown in FIG. 5, the ultrasonic probe 1 is connected to a apparatus 22 of the ultrasonic diagnostic apparatus via a cable 21. An ultrasonic probe controller (not shown) for permitting the ultrasonic probe 1 to transmit and receive ultrasonic signals, a display 23, etc., are housed in the apparatus 22 of the ultrasonic diagnostic apparatus.

As described above, the array-type ultrasonic probe according to the embodiment described above comprises a plurality of channels arranged with spaces and each including a piezoelectric element and at least three acoustic matching layers formed on the piezoelectric element, a backing member, and an acoustic lens that is formed in a manner to cover at least the upper surface of the uppermost acoustic matching layer included in each channel. The piezoelectric element included in each channel is mounted on the backing member, and trenches corresponding to the spaces between the adjacent channels are formed in a surface region of the backing member. The uppermost acoustic matching layer contains a denatured polyether resin and an another resin having an acoustic impedance higher than that of the denatured polyether resin. Also, the uppermost acoustic matching layer exhibits an acoustic impedance of 1.6 to 2.5 MRayls at 25° C. The array-type ultrasonic probe comprising the uppermost acoustic matching layer of the composition described above produces prominent effects as summarized:

(1) Since the uppermost acoustic matching layer exhibits a low attenuation and an appropriate acoustic impedance, it is possible to provide an array-type ultrasonic probe of a high performance capable of effectively transmitting and receiving the energy of the ultrasonic wave.

(2) Since the uppermost acoustic matching layer exhibits an excellent dicing machinability it is possible to form accurately a channel having a desired width by the dicing process using, for example, a dicing blade. As a result, the crosstalk between the adjacent channels can be suppressed so as to provide an array-type ultrasonic probe exhibiting a high resolution.

(3) The uppermost acoustic matching layer is excellent in its heat resistance and exhibits a high adhesivity to the silicone-based adhesive layer and the epoxy-based adhesive layer interposed between the upper and lower layers (i.e., between the acoustic lens and the acoustic matching layer formed below the acoustic lens). As a result, it is possible to prevent the peeling between the acoustic lens and the uppermost acoustic matching layer and between the uppermost acoustic matching layer and the lower acoustic matching layer even if the acoustic matching layer is heated or a mechanical pressure is applied to the acoustic matching layer in accordance with the absorption and attenuation of the energy of the ultrasonic wave. It follows that it is possible to provide an array-type ultrasonic probe having a high reliability over a long time, in which the channels are allowed to exhibit uniform sensitivity.

It should be noted in particular that the uppermost acoustic matching layer, which contains epoxy resin particles as the another resin dispersed in the denatured polyether resin matrix in an amount of 10 to 60% by volume based on the sum of the matrix and the epoxy resin particles, permits further improving the adhesivity of the uppermost acoustic matching layer to the epoxy-based adhesive. Also, the machinability of the uppermost acoustic matching layer is further improved in the stage of the dicing treatment.

It should also be noted that it is possible for the polymer alloy forming the uppermost acoustic matching layer to further contain an inorganic filler having a density not higher than 6 g/cm³. The inorganic filler is added to the polymer alloy in an amount not larger than 30% by volume based on the sum of the polymer alloy and the inorganic filler. The uppermost acoustic matching layer formed of the polymer alloy containing the inorganic filler exhibits a attenuation, which is measured at 5 MHz, not higher than 8 dB/cm MHz, and an attenuation figure of merits (which is the product between the attenuation and acoustic velocity), which is not larger than 1,500, so as to further improve the transmitting-receiving performance of the energy of the ultrasonic wave. In addition, the particular uppermost acoustic matching layer exhibits further improvements in the machinability in the stage of the dicing treatment and in the mechanical strength.

Further, in the case of using the fibrous inorganic filler, it is possible to improve further the attenuation and the machinability of the acoustic matching layer in the stage of the dicing treatment as well as the mechanical strength of the acoustic matching layer.

The ultrasonic diagnostic apparatus according to the embodiment comprises an array-type ultrasonic probe, which exhibits low crosstalk, high performance, and high reliability, and, thus, the ultrasonic diagnostic apparatus improves the quality of a tomographic image and improves sensitivity.

The present invention will now be described more in detail with reference to the Examples given below.

### EXAMPLE 1

Sufficiently mixed were the raw materials comprising 100 parts by weight of a liquid denatured polyether resin (silyl resin manufactured by Kaneka K.K.), 50 parts by weight of Epicoat 328 (trade name of a liquid bisphenol A type epoxy resin manufactured by Japan Epoxy Resin Inc.), which was used as a second resin, 1 part by weight of a bisphenol A type antioxidant, 1 part by weight of diphenyl silane diol, 2 parts by weight of an organic tin compound, 5 parts by weight of 2,4,6-tris (dimethylamino methyl) phenol epoxy resin, which was used as a curing agent, and 0.4 part by weight of distilled water. The mixture was put in a container made of polyethylene and de-gassed, followed by curing the mixture at 50° C. for 72 hours so as to obtain a cured material (which was used for manufacturing a third acoustic matching layer).

### EXAMPLES 2 TO 10, REFERENCE EXAMPLES 1 TO 3, AND COMPARATIVE EXAMPLES 1 TO 3

Fifteen kinds of the materials for forming the third acoustic matching layer were obtained as in Example 1, except that the mixing ratio of the base resin of the liquid denatured polyether resin (silyl resin manufactured by Kaneka K.K.) to the second resin of Epicoat 328 (trade name of a liquid bisphenol A type epoxy resin manufactured by Japan Epoxy Resin Inc.) was changed as shown in Table 1 and that a powdery inorganic filler and a fibrous inorganic filler were further added to the raw materials used in Example 1 as shown in Table 1.

Incidentally, Hiker-RLP (trade name of a carboxylic group terminal liquid acrylonitrile/butadiene liquid rubber (CTBN) manufactured by Ube Kosan K.K.) was used as the acrylic resin shown in Table 1. Also, the powdery inorganic fillers shown in Table 1 include a tungsten powder, which was in the form of spherical particles having an average particle diameter of 1 μm, a silica powder, which was in the form of spherical particles having an average particle diameter of 20 nm, a zinc oxide powder, which was in the form of spherical particles having an average particle diameter of 200 nm, a carbon powder, which was in the form of spherical particles having an average particle diameter of 20 nm, and a titanium oxide powder, which was in the form of spherical particles having an average particle diameter of 50 nm. On the other hand, the fibrous inorganic filler shown in Table 1 includes a carbon fiber having a diameter of 7 μm and an average length of 100 μm, a glass fiber having a diameter of 5 μm and an average length of 100 μm, and a silica fiber having a diameter of 8 μm and an average length of 100 μm.

The density, acoustic velocity, acoustic impedance (AI), attenuation, machinability heat resistance and adhesivity were measured by the methods given below in respect of the materials of the third acoustic matching layer for each of Examples 1 to 10, Reference Examples 1 to 3 and Comparative Examples 1 to 3:

1) Density

The density was obtained from a disk made from the material of the third acoustic matching layer. For measuring the density, the weight of the sample was measured within the air and within water at 25° C., and the density was measured by the Archimedean method.

2) Acoustic Velocity and Attenuation

A test piece 30 mm wide, 30 mm long and 1 mm thick was prepared by using the material of the third acoustic matching layer. The acoustic velocity and the attenuation of the test piece were measured within water of 25° C. by using a measuring probe of 5 MHz. Ultrasonic signals were transmitted from an ultrasonic probe to stainless steel held stationary within water and to a sample that was also held stationary, and the echo signals were measured.

The acoustic velocity was obtained from the difference in time between the echo signals, which was caused by the presence of the sample, and from the thickness of the sample. The acoustic velocity (C) was calculated by the formula given below by utilizing the difference in time of the transmission waveform between water and the sample, with the acoustic velocity of water at each temperature set as the reference.

C=C₀/[L−C₀(Δt/d)]

where C₀ denotes the acoustic velocity of water, L denotes the distance between the ultrasonic probe and the sample (object to be measured), d denotes the thickness of the sample, and Δt denotes the difference in time of the zero-cross point after the transmission waveforms of water and the sample passed through the initial peaks.

Further, the attenuation was obtained by a prescribed method based on the difference in intensity between the echo signals in water of 25° C., which was caused by the presence of the sample, and also based on the thickness of the sample.

3) Acoustic Impedance (AI)

The acoustic impedance (AI) was obtained as the product of the density and the acoustic velocity, which were measured as described above.

4) Machinability

A test piece 30 mm wide, 30 mm long and 1 mm thick was prepared from the material of the third acoustic matching layer. The test piece was engraved at a pitch of 100 μm to reach a depth of 200 μm by using a diamond blade having a thickness of 50 μm, followed by rotating the test piece by 90° so as to engrave again the test piece at a pitch of 100 μm to reach a depth of 200 μm. The remaining portion (50 μm×50 μm) after the engraving step was observed with a microscope. In this observation, the machinability was evaluated from the fall and linearity of the third acoustic matching layer.

The machinability was evaluated as four stages of A, B, C, and D given below:

A: The remaining piece sized at 50 μm square was quite free from problems.

B: A defect not larger than 2% was observed in the remaining piece sized at 50 μm square.

C: A defect not larger than 10% was observed in the remaining piece sized at 50 μm square.

D: A defect exceeding 10% was observed in the remaining piece sized at 50 μm square.

5) Heat Resistance

A test piece 25 mm wide, 100 mm long and 1.6 mm thick was prepared from the material of the third acoustic matching layer. The test piece was attached to a glass fiber reinforced epoxy substrate (FR4) in accordance with the method specified in JIS-C6471 8.1 by using an epoxy adhesive, followed by curing the adhesive at 60° C. for 24 hours and, then, at 125° C. for one hour. Then, the test piece was pulled at a velocity of 30 cm/min by using a Tensilon type tensilometer so as to obtain the tensile shear strength. Ten test pieces were used for the test and the average value thereof was obtained.

The heat resistance was evaluated as five stages of A, B, C, D and E given below:

A: The shear strength after the heat treatment was not lower than 3.0 N/mm²;

B: The shear strength after the heat treatment was not lower than 2.0 N/mm²;

C: The shear strength after the heat treatment was not lower than 1.0 N/mm²;

D: The shear strength after the heat treatment was not lower than 0.5 N/mm²;

E: The shear strength after the heat treatment was lower than 0.5 N/mm².

6) Adhesivity

A test piece 25 mm wide, 100 mm long and 1.6 mm thick was prepared from the material of the third acoustic matching layer. The test piece was attached to a silicone rubber plate, which was equal to the material used for preparing the acoustic lens, having a density of 1.5 g/cm³ and a thickness of 5 mm (an aluminum sheet having a thickness of 5 mm was attached to the back surface of the silicone rubber plate) in accordance with the method specified in JIS-C6471 8.1 by using an silicone rubber-based adhesive [registered trademark of “Cemedine Super X No. 8008 clear”], followed by curing the adhesive at 60° C. for 24 hours. Then, the test piece was pulled at a velocity of 30 cm/min by using a Tensilon type tensilometer so as to obtain the peeling strength. Ten test pieces were used for the test and an average value thereof was obtained.

The adhesivity was evaluated as five stages of A, B, C, D and E given below:

A: The peeling strength after the heat treatment was not lower than 1.0 N/mm²;

B: The peeling strength after the heat treatment was not lower than 0.75 N/mm²;

C: The peeling strength after the heat treatment was not lower than 0.5 N/mm²;

D: The peeling strength after the heat treatment was not lower than 0.3 N/mm²;

E: The peeling strength after the heat treatment was lower than 0.3 N/mm².

Table 2 shows the experimental data.

TABLE 1

| | Composition of third or fourth acoustic matching layer | | | | | | | |
|---|---|---|---|---|---|---|---|---|
| | | | | | Powdery | | Fibrous | |
| | Base resin | | Another resin | | inorganic filler | | inorganic filler | |
| | Material | Amount (vol %) | Material | Amount (vol %) | Material | Amount (vol %) | Material | Amount (vol %) |
| Example 1 | Denatured | 50 | Epoxy | 50 | — | — | — | — |
| | polyether | | resin | | | | | |
| Reference | Denatured | 95 | Epoxy | 5 | — | — | — | — |
| Example 1 | polyether | | resin | | | | | |
| Reference | Denatured | 20 | Epoxy | 80 | — | — | — | — |
| Example 2 | polyether | | resin | | | | | |
| Reference | Denatured | 48 | Epoxy | 48 | Tungsten | 4 | — | — |
| Example 3 | polyether | | resin | | | | | |
| Example 2 | Denatured | 50 | Epoxy | 30 | Silica | 20 | — | — |
| | polyether | | resin | | | | | |
| Example 3 | Denatured | 72 | Epoxy | 18 | Silica | 5 | Carbon | 5 |
| | polyether | | resin | | | | | |
| Exampe 4 | Denatured | 60 | Epoxy | 30 | Silica | 5 | Glass | 5 |
| | polyether | | resin | | | | | |
| Example 5 | Denatured | 45 | Epoxy | 45 | Zinc | 5 | Glass | 5 |
| | polyether | | resin | | oxide | | | |
| Example 6 | Denatured | 45 | Epoxy | 45 | Carbon | 5 | Carbon | 5 |
| | polyether | | resin | | | | | |
| Example 7 | Denatured | 80 | Epoxy | 10 | Silica | 5 | Glass | 5 |
| | polyether | | resin | | | | | |
| Example 8 | Denatured | 45 | Acrylic | 45 | Titanium | 5 | Glass | 5 |
| | polyether | | resin | | oxide | | | |
| Example 9 | Denatured | 45 | Acrylic | 45 | Silica | 5 | Carbon | 5 |
| | polyether | | resin | | | | | |
| Example 10 | Denatured | 65 | Epoxy | 20 | Aluminum | 5 | Silica | 10 |
| | polyether | | resin | | oxide | | | |
| Comparative | Polyurethane | 90 | — | — | Silica | 10 | — | — |
| Example 1 | | | | | | | | |
| Comparative | Silicone | 90 | — | — | Silica | 10 | — | — |
| Example 2 | resin | | | | | | | |
| Comparative | Epoxy | 90 | — | — | Silica | 10 | — | — |
| Example 3 | resin | | | | | | | |

TABLE 2

| | Characteristics of third or fourth acoustic matching layer | | | | | | |
|---|---|---|---|---|---|---|---|
| | | Acoustic | | | | | |
| | Density | velocity | | Damping factor | | Heat | |
| | (g/cm³) | (m/s) | AI (MRayls) | (dB/cmMHz) | Machinability | resistance | Adhesivity |
| Example 1 | 1.05 | 1620 | 1.70 | 3.5 | B | B | B |
| Reference | 1.01 | 1100 | 1.11 | 3.0 | D | C | E |
| Example 1 | | | | | | | |
| Reference | 1.08 | 2400 | 2.59 | 16.0 | B | C | A |
| Example 2 | | | | | | | |
| Reference | 1.78 | 1500 | 2.67 | 17.8 | C | A | A |
| Example 3 | | | | | | | |
| Example 2 | 1.27 | 1380 | 1.75 | 7.4 | B | A | A |
| Example 3 | 1.13 | 1450 | 1.64 | 4.5 | A | A | A |
| Example 4 | 1.14 | 1440 | 1.64 | 4.8 | A | A | A |
| Example 5 | 1.33 | 1500 | 2.00 | 5.0 | A | A | A |
| Example 6 | 1.15 | 1590 | 1.83 | 4.5 | A | A | A |
| Example 7 | 1.12 | 1480 | 1.66 | 4.0 | A | A | A |
| Example 8 | 1.30 | 1650 | 2.15 | 7.0 | A | B | B |
| Example 9 | 1.13 | 1790 | 2.03 | 6.5 | A | B | B |
| Example 10 | 1.37 | 1700 | 2.33 | 7.8. | A | A | B |
| Comparative | 1.09 | 1820 | 1.98 | 14.2 | D | D | D |
| Example 1 | | | | | | | |
| Comparative | 1.09 | 1050 | 1.14 | 4.0 | D | A | D |
| Example 2 | | | | | | | |
| Comparative | 1.21 | 2800 | 3.39 | 6.5 | C | B | B |
| Example 3 | | | | | | | |

As apparent from Tables 1 and 2 given above, the third acoustic matching layer for each of Examples 1 to 10 was low in the attenuation and excellent in each of the machinability heat resistance and the adhesivity, though the acoustic impedance (AI) of the third acoustic matching layer was low, i.e., 1.6 to 2.5 MRayls.

On the other hand, the third acoustic matching layer for each of Reference Examples 1 to 3, which exhibited the acoustic impedance (AI) failing to fall within the range of 1.6 to 2.5 MRayls in spite of the same composition, exhibited a large attenuation or was found to be poor in any of the machinability the heat resistance and the adhesivity, failing to satisfy all of these characteristics.

Also, the third acoustic matching layer for each of Comparative Examples 1 to 3, in which polyurethane, silicone rubber and epoxy resin were used as the base materials, respectively, was found to have a large attenuation or found to be poor in any of the machinability the heat resistance and the adhesivity, failing to satisfy all of these characteristics.

As pointed out above, it has been confirmed that the third acoustic matching layer for each of Examples 1 to 10 was low in the attenuation and excellent in each of the machinability heat resistance and adhesivity, though the acoustic impedance (AI) of the third acoustic matching layer was low, i.e., 1.6 to 2.5 MRayls. An array-type ultrasonic probe that was assembled as described below by using the third acoustic matching layer noted above was found to be capable of effectively transmitting-receiving the energy of the ultrasonic wave, to be uniform in the sensitivity of the channels, and to be capable of suppressing the crosstalk between the adjacent channels so as to achieve high resolution and high reliability over a long time.

To be more specific, a piezoelectric element having a thickness of 400 μm was placed on a backing member with an epoxy resin-based adhesive interposed therebetween, followed by disposing a first acoustic matching layer having a thickness of 420 μm on the piezoelectric element with an epoxy resin-based adhesive interposed therebetween. Then, a second acoustic matching layer having a thickness of 200 μm was placed on the first acoustic matching layer with an epoxy resin-based adhesive interposed therebetween, followed by placing a third acoustic matching layer having a thickness of 150 μm on the second acoustic matching layer, with an epoxy resin-based adhesive interposed therebetween, so as to obtain a laminated structure. The backing member noted above was prepared by adding ferrite to chloroprene rubber having an acoustic impedance (AI) of 4 MRayls. The first acoustic matching layer noted above was formed of a borosilicate glass having an acoustic impedance (AI) of 12 MRayls. The second acoustic matching layer noted above, which had an acoustic impedance (AI) of 5.0 MRayls, was formed by adding a zinc oxide powder to an epoxy resin in an amount of 20% by volume. Further, the third acoustic matching layer noted above had an acoustic impedance (AI) for Examples 1 to 10 shown in Table 2. The laminate structure was heated at 120° C. for about one hour under pressure so as to cure the adhesive and, thus, to bond the constituting members of the laminated structure to each other. Incidentally, the piezoelectric element was prepared by forming the first and second electrodes each formed of Ni on both surfaces of a piezoelectric body consisting of a PZT-based piezoelectric ceramic material. Then, a dicing treatment was applied to the laminated structure by using a diamond blade having a width of 50 μm such that the laminated structure was cut from the third acoustic matching layer toward the backing member in a width of 200 μm. In this dicing treatment, the backing member was cut in a depth of 200 μm, and 200 channels were formed in total, each channel being sized at 200 μm×2 columns. In the next step, a liquid silicone rubber was loaded in the space between the adjacent channels and cured at 125° C. for one hour. Then, an acoustic lens having an acoustic impedance (AI) of 1.5 MRayls and formed of a silicone rubber was fixed to the upper surface of each channel, with a denatured silicone rubber-based adhesive interposed therebetween. Finally, the backing member, the plural channels and the acoustic lens were housed in a case. Also housed in the case were a control circuit for controlling the driving timing of the piezoelectric element included in each channel and a signal processing circuit including an amplifying circuit for amplifying the signal received by the piezoelectric element 6, thereby assembling an array-type ultrasonic probe of 3.5 MHz.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An array-type ultrasonic probe, comprising:
a member;
a plurality of channels arranged on the member with spaces and each having a piezoelectric element and at least three acoustic matching layers formed on the piezoelectric element; and
an acoustic lens formed in a manner to cover at least the surface of the uppermost acoustic matching layer of each channel,
wherein the uppermost acoustic matching layer contains a denatured polyether resin and an another resin having an acoustic impedance higher than that of the denatured polyether resin, the uppermost acoustic matching layer having an acoustic impedance of 1.6 to 2.5 MRayls at 25° C.

2. The probe according to claim 1, wherein each of the plural channels has a width of 50 to 200 μm.

3. The probe according to claim 1, wherein trenches corresponding to the spaces between the adjacent channels are formed in the member.

4. The probe according to claim 1, wherein the first resin is provided by an epoxy resin.

5. The probe according to claim 1, wherein the uppermost acoustic matching layer contains the denatured polyether resin and epoxy resin particles dispersed as the another resin in the denatured polyether resin in an amount of 10 to 60% by volume based on the sum of the denatured polyether resin and the epoxy resin particles and has a longitudinal wave acoustic velocity of 1,300 to 1,800 m/s at 25° C.

6. The probe according to claim 5, wherein the epoxy resin particles have an average particle diameter of 1 to 5 μm.

7. The probe according to claim 1, wherein the uppermost acoustic matching layer further contains at least one inorganic filler selected from the group consisting of a powdery inorganic filler having a density not higher than 6 g/cm³ and a fibrous inorganic filler in an amount not larger than 30% by volume based on the sum of the denatured polyether resin, the another resin and the inorganic filler.

8. The probe according to claim 7, wherein the uppermost acoustic matching layer has a attenuation measured at 5 MHz, which is not larger than 8 dB/cm MHz, and a damping capacity index, which is the product between the attenuation and the acoustic velocity, which is not larger than 1,500 m/s·dB/mm MHz.

9. The probe according to claim 7, wherein the powdery inorganic filler is at least one powder selected from the group consisting of zinc oxide powder, zirconium oxide powder, alumina powder, silica powder such as aerosil silica, titanium oxide powder, silicon carbide powder, aluminum nitride powder, carbon powder and boron nitride powder.

10. The probe according to claim 7, wherein the powdery inorganic filler has an average particle diameter not larger than 0.5 μm.

11. The probe according to claim 7, wherein the fibrous inorganic filler is at least one fiber selected from the group consisting of a carbon fiber, a silicon carbide fiber, a zinc oxide fiber, an alumina fiber and a glass fiber.

12. The probe according to claim 7, wherein the fibrous inorganic filler has a diameter not larger than 10 μm and a length at least 5 times as much as the diameter.

13. The probe according to claim 1, wherein the acoustic lens is bonded on the uppermost acoustic matching layer each of the channels by using a rubber-based adhesive having an acoustic impedance of 1.3 to 1.8 MRayls at 25° C.

14. An array-type ultrasonic probe, comprising:
a member;
a plurality of channels arranged on the member with spaces and each having a piezoelectric element and at least three acoustic matching layers formed on the piezoelectric element; and
an acoustic lens formed in a manner to cover at least the surface of the uppermost acoustic matching layer of each channel,
wherein the lowermost acoustic matching layer in contact with the piezoelectric element has an acoustic impedance of 10 to 15 MRayls at 25° C., the intermediate acoustic matching layer has an acoustic impedance of 2.7 to 8 MRayls at 25° C., and the uppermost acoustic matching layer in contact with the acoustic lens contains a denatured polyether resin and an another resin having an acoustic impedance higher than that of the denatured polyether resin, the uppermost acoustic matching layer having an acoustic impedance of 1.6 to 2.5 MRayls at 25° C.

15. An array-type ultrasonic probe, comprising:
a member;
a plurality of channels arranged on the member with spaces and each having a piezoelectric element and at least four acoustic matching layers formed on the piezoelectric element; and
an acoustic lens formed in a manner to cover at least the surface of the uppermost acoustic matching layer of each channel,
wherein the lowermost acoustic matching layer in contact with the piezoelectric element has an acoustic impedance of 14 to 20 MRayls at 25° C., the second acoustic matching layer in contact with the lowermost acoustic matching layer has an acoustic impedance of 7 to 12 MRayls at 25° C., the third acoustic matching layer in contact with the second acoustic matching layer has an acoustic impedance of 3 to 5 MRayls at 25° C., and the uppermost acoustic matching layer in contact with the acoustic lens contains a denatured polyether resin and an another resin having an acoustic impedance higher than that of the denatured polyether resin, the uppermost acoustic matching layer having an acoustic impedance of 1.6 to 2.5 MRayls at 25° C.

16. An ultrasonic diagnostic apparatus, comprising:
an array-type ultrasonic probe defined in claim 1; and
an ultrasonic probe controller connected to the ultrasonic probe through a cable.
